(19) United States
(12) Patent Application Publication
(10) Pub. No.: US 2007/0026046 A1
(43) Pub. Date: Feb. 1, 2007

(54) Neoglycopolymer-cross-linked biopolymer matrix

(75) Inventors: Deryn Fogg, Ottawa (CA); Yuwen Liu, Pleasenton, CA (US); May Griffith, Carp (CA)

Correspondence Address: FOLEY & LARDNER LLP, 111 HUNTINGTON AVENUE, 26TH FLOOR, BOSTON, MA, 02199-7610, US

(73) Assignees: University of Ottawa; Ottawa Health Research Institute

(21) Appl. No.: 11/442,755

(22) Filed: May 30, 2006

Related U.S. Application Data
(60) Provisional application No. 60/684,988, filed on May 27, 2005.

(51) Int. Cl. A61K 38/39; A61K 9/14; A61F 2/00

(52) U.S. Cl. 424/427; 424/486; 514/2

(57) ABSTRACT

The present application provides a cross-linked biopolymer matrix, or scaffold, comprising a biopolymer and cross-linked with a neoglycopolymer. A specific example of a biopolymer matrix according to this invention comprises collagen as the biopolymer. Also provided is a method for producing the cross-linked biopolymer matrix and methods of use thereof.

### RELATED APPLICATION DATA

This application claims the benefit of U.S. Provisional Patent Application No. 60/684,988 filed May 27, 2005, the entire contents of which are hereby incorporated by reference.

### FIELD OF THE INVENTION

This invention relates to generally to the field of biopolymer-based matrices and, more specifically, to the field of cross-linked biopolymer-based matrices or scaffolds.

### BACKGROUND OF THE INVENTION

Tissue engineering strategies directed at replicating key properties of biosynthetic matrices have generated much interest for their potential to alleviate issues of organ failure and donor organ shortages (Lee, K. Y.; Mooney, D. J. Chem. Rev. 2001, 101, 1869-1879; and Langer, R.; Vacanti, J. P. Science 1993, 260, 920-926). Synthetic polymer scaffolds have received considerable attention (Atala, A.; Mooney, D. J.; Vacanti, J. P.; Langer, R. S., Ed. Synthetic Biodegradable Polymer Scaffolds. Birkhauser: Boston, 1997; Shoichet, M. S.; Hubell, J. A., Ed. Polymers for Tissue Engineering. VSP: Utrecht, Netherlands, 1998; and Hoffman, A. S. Adv. Drug. Deliv. Rev. 2002, 54, 3-12), and can be formulated to exhibit predetermined physical characteristics such as gel strength, as well as biological characteristics such as degradability. However, reports that synthetic analogues of natural polymers, such as polylysine, poly(ethylene imine), and the like, can exhibit cytotoxic effects (Lynn & Langer, J. Amer. Chem. Soc., 122:10761-10768 (2000)) have lead to the development of alternative synthetic polymers for tissue engineering applications. Natural bio-polymers such as collagens, fibrin, alginates and agarose are known to be non-cytotoxic and to support over-growth, in-growth and encapsulation of living cells. Matrices derived from natural polymers, however, are generally insufficiently robust for transplantation. Collagen-based “tissue equivalents” provide an attractive alternative to synthetic polymers (Griffith, M.; Osborne, R.; Munger, R.; Xiong, X.; Doillon, C. J.; Laycock, N. L. C.; Hakim, M.; Song, Y.; Watsky, M. A. Science 1999, 286, 2169-2172; Weinberg, C. B.; Bell, E. Science 1986, 231, 397-400; Bell, E.; Ehrlich, H. P.; Buttle, D. J.; Nakatsuji, T. Science 1981, 211, 1052; and Girton, T. S.; Oegema, T. R.; Tranquillo, R. T. J. Biomed. Mat. Res. 1999, 46, 87-92). For instance, cross-linked collagen matrices can be used for corneal regeneration (Shimmura, S.; Doillon, C., J.; Griffith, M.; Nakamura, M.; Gagnon, E.; Usui, A.; Shinozaki, N.; Tsubota, K. Cornea 2003, 22, S81-8; Li, F.; Carlsson, D.; Lohmann, C.; Suuronen, E.; Vascotto, S.; Kobuch, K.; Sheardown, H.; Munger, R.; Nakamura, M.; Griffith, M. Proc. Nat. Acad. Sci. USA 2003, 100, 15346-15351; International Patent Application WO 2004/014969 and Li, F.; Griffith, M.; Li, Z.; Tanodekaew, S.; Sheardown, H.; Hakim, M.; Carlsson, D. J. Biomaterials 2005, 26, 3093-3104).

Collagen comprises about one third of the total protein in mammalian organisms and is the main constituent of the extracellular matrices (ECM) of mammalian tissues, such as skin and connective tissue. It is a natural cell substrate that promotes cell spreading and binding of other ECM components, providing a multipurpose scaffold that can aid in tissue reconstruction. Collagen is composed of three chains that form a triple helix. The amino acid sequence of the chains is mostly a repeating structure with glycine in every third postion and proline or hydroxyproline frequently preceding the glycine residues. Collagen molecules are typically cross-linked together, forming fibrils. In the synthesis of collagen-based scaffolds, chemical cross-linking can be used to improve mechanical properties, as well as stability toward enzymatic degradation (Rault, I.; Frei, V.; Herbage, D.; Abdul-Malak, N.; Huc, A. J. Mater. Sci. Mater. Med. 1996, 7, 215-21; and Weadock, K.; Olson, R. M.; Silver, F. H. Biomat. Med. Dev. Art. Org. 1984 11 293-318).

Glutaraldehyde and carbodiimides are currently among the most widely used collagen-cross-linking agents (Lee, K. Y.; Mooney, D. J. Chem. Rev. 2001, 101, 1869-1879; and Rault, I.; Frei, V.; Herbage, D.; Abdul-Malak, N.; Huc, A. J. Mater. Sci. Mater. Med. 1996, 7, 215-21), despite a susceptibility of the resulting materials to calcification (Nimni, M. E. J. Biomed. Mat. Res. 1987, 21, 741-771) and potential for local cytotoxicity (Rault, I.; Frei, V.; Herbage, D.; Abdul-Malak, N.; Huc, A. J. Mater. Sci. Mater. Med. 1996, 7, 215-21). Carbohydrates are non-cytotoxic and can also be used to cross-link collagen. Using carbohydrates, the key initial step involves formation of Schiff base products via condensation of aldehyde groups of the ring-open sugars with amine groups present in lysine and hydroxylysine residues of the collagen (Girton, T. S.; Oegema, T. R.; Tranquillo, R. T. J. Biomed. Mat. Res. 1999, 46, 87-92; and Ohan, M. P.; Weadock, K. S.; Dunn, M. G. J. Biomed. Mat. Res. 2002, 60, 384-391). Glucose and ribose are examples of carbohydrates that have been used to cross-link collagen. However, the reaction using glucose and ribose to cross-link collagen is very slow and can take from 2 weeks to over 3 months.

Thus, it is desirable to provide other carbohydrate-based cross-linkers which could overcome these disadvantages.

Certain carbohydrate-functionalized polymers are known. Carbohydrate-functionalized polymers, sometimes referred to as neoglycopolymers, are synthetic polymers comprising pendant carbohydrate groups, wherein the carbohydrate groups are typically monosaccharides. For instance, Kiessling and her co-workers constructed a polymer with pendant galactose-3,6-disulfate, and investigated its use as a potential P-selectin-mediated cell adhesion blocker (Manning, D. D.; Hu, X.; Beck, P.; Kiessling, L. L. J. Am. Chem. Soc. 1997, 119 3161-2). However, use of neoglycopolymers as collagen cross-linking agents is not known.

Ring-opening metathesis polymerization (ROMP) techniques have been used previously for construction of neoglycopolymers (Mortell, K. H.; Gingras, M.; Kiessling, L. L. J. Am. Chem. Soc. 1994, 116, 12053-4; Fraser, C.; Grubbs, R. H. Macromolecules 1995, 28, 7248-55; Nomura, K.; Schrock, R. R. Macromolecules 1996, 29, 540-5; Nomura, K.; Sakai, I.; Imanishi, Y.; Fujiki, M.; Miyamoto, Y. Macromol. Rapid Commun. 2004, 25, 571-576; and Miyamoto, Y.; Fujiki, M.; Nomura, K. J. Polym. Sci., Part A: Polym. Chem. 2004, 42, 4248-4265). The ROMP methodology involves subjecting a closed ring containing an olefinic functional group to a ring-opening polymerization reaction which results in an unsaturated polymer. Reduction of the ROMP polymers is often important in order to retain control of polymer microstructure (Schnabel, W. Polymer Degradation: Principles and Practical Applications, 1993), producing the corresponding saturated polymer products. Polymerization and post-polymerization hydrogenation (by, for example, diimide reduction) (Kanai, M.; Mortell, K. H.; Kiessling, L. L. J. Am. Chem. Soc. 1997, 119, 9931-9932) are typically carried out in two separate stages. ROMP polymerization and subsequent hydrogentation reactions, which utilize the same catalyst are know as “tandem ROMP-hydrogenation” reactions, in which a single catalyst effect both polymerization and hydrogenation/hydrogenolyis. It has been shown (Drouin, S. D.; Yap, G. P. A.; Fogg, D. E. Inorg. Chem. 2000, 39, 5412-5414; Drouin, S. D.; Zamanian, F.; Fogg, D. E. Organometallics 2001, 20, 5495-5497; McLain, S. J.; McCord, E. F.; Arthur, S. D.; Hauptman, E.; Feldman, J.; Nugent, W. A.; Johnson, L. K.; Mecking, S.; Brookhart, M. Polym. Mater. Sci. Eng. 1997, 76, 246-247; and Bielawski, C. W.; Louie, J.; Grubbs, R. H. J. Am. Chem. Soc. 2000, 122, 12872-12873) that tandem processes of Ru-catalyzed ROMP and hydrogenation can provide efficient routes to saturated polyolefins (Fogg, D. E.; dos Santos, E. N. Coord. Chem. Rev. 2004, 248, 2365-2379).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a cross-linked biopolymer matrix or scaffold. Thus, in one aspect the present invention provides a method for producing a biopolymer matrix, comprising the step of cross-linking a biopolymer and a neoglycopolymer in an aqueous medium. In accordance with one embodiment of the present invention, the biopolymer is collagen.

In another aspect the present invention provides a biopolymer matrix comprising a biopolymer and a neoglycopolymer, wherein the biopolymer and the neoglycopolymer are cross-linked. In accordance with one embodiment of the present invention, the biopolymer is collagen.

In a further aspect, the invention provides use of a neoglycopolymer-cross-linked biopolymer matrix as an artificial cornea or part thereof.

### BRIEF DESCRIPTION OF THE FIGURES

FIG. 1 depicts a schematic representation of aldehyde generation and cross-linking of collagen to form a matrix according to one embodiment of the present invention.

FIG. 2 depicts solid-state ¹³C NMR spectra of (a) neoglycopolymer (n=50); (b) Coll-neo50-2; and (c) collagen.

FIG. 3 provides graphical representations of the results of measuring the tensile strength (A), average break strain (B) and moduli (C) of hydrogels according to specific embodiments of the present invention.

FIG. 4 is a photograph image of a Coll-neo50-2-Rim construct (A: suture; B: corneal rim; C: glass support; and D: Coll-neo50-2 hydrogel).

FIG. 5 depicts DSC heating thermograms of a collagen solution (13.7% w/w un-cross-linked porcine type I collagen solution) and Coll-neoSO hydrogels.

FIG. 6 depicts in vitro biodegradation kinetics of collagen hydrogels corsslinked with different ratios of neoglycopolymer (n=50). The arrow indicates the complete degradation.

FIG. 7 depicts rat subcutaneous implantation of a hydrogel according to a specific embodiment of the present invention.

FIG. 8 provides images of human corneal epithelial growth on the surface of Coll-neo50-2 hydrogel (A) and control (culture plate) (B) at day 6 post-seeding.

FIG. 9 is a graphical representation of quantitative analysis of human corneal epithelial cell growth on Coll-neo50-2 hydrogels. Each assay was performed in triplicate. The control is culture plate surface.

FIG. 10 depicts growth of chick dorsal root ganglion on the surface of Coll-neo50-2 hydrogel. Neurites are indicated by arrows.

### DETAILED DESCRIPTION OF THE INVENTION

The present invention provides a neoglycopolymer-cross-linked biopolymer matrix or hydrogel (also referred to herein as a scaffold), which is formed by cross-linking a bio-polymer with a neoglycopolymer. The bio-polymer can be in its naturally-occurring form, or it can be derivatised to facilitate cross-linking with the neoglycopolymer. The matrix is robust, biocompatible and non-cytotoxic. The matrix can be formed in aqueous solution and can be tailored to further comprise one or more bioactive agents such as growth factors, retinoids, cell adhesion factors, enzymes, peptides, proteins, nucleotides, drugs, and the like. The bioactive agent can be covalently attached to the synthetic polymer, or it can be encapsulated and dispersed within the final matrix depending on the end use demands for the matrix. The matrix can also comprise cells encapsulated and dispersed therein, which are capable of proliferation and/or diversification upon deposition of the matrix in vivo.

In one embodiment of the present invention, the biopolymer based matrix supports cell growth. In another embodiment of the invention, the bio-synthetic matrix supports nerve in-growth.

The biopolymer based matrix can be tailored for specific applications. For example, the matrix can be used in tissue engineering applications and can be pre-formed into a specific shape for this purpose.

In order to be suitable for in vivo implantation for tissue engineering purposes, the biopolymer matrix must maintain its form at physiological temperatures, be substantially insoluble in water, be adequately robust, and support the growth of cells. Depending on the application of the matrix, it may also be desirable for the matrix to support the growth of nerves.

Neoglycopolymer

The neoglycopolymers used in accordance with the present invention are synthetic polymers comprising pendant carbohydrate groups. The carbohydrate groups are typically monosaccharides, though disaccharides and oligosaccharide chains can also be used. The saccharides should be reducing sugars (also known as oglycosides), meaning that they undergo keto-enol tautomerization (sugar ring opens and closes) in aqueous solutions. Examples of carbohydrate pendant groups include, but are not limited to, galactose, ribose, glucose, glycerose, threose, erythrose, lyxose, xylose, arabinose, allose, altrose, manno se, gulose, idose, talose, disaccharides and oligosaccharide chains, both native and derivatized molecules, and combinations thereof.

The neoglycopolymer backbone can be made from polymerized norbornene, said norbornenes being derivatized with the carbohydrate molecules.

The neoglycopolymer can contain pendant carbohydrate groups at every monomer or only at select monomers. Furthermore, on a given monomer there may be one or more saccharide molecules. For instance, the monomer could be mono or bi-substituted, for example, by galactose.

Though the present invention is described in relation to a synthetic polymer made with pendant carbohydrate groups, it would be obvious to one skilled in the art that use of a carbohydrate pendant group is not essential. The synthetic polymer can instead contain other pendant groups, so long as the pendant group contained a functional group, e.g., an aldehyde, capable of reacting with amine groups on a biopolymer.

The overall hydrophilicity of the neoglycopolymer is controlled to confer water solubility at temperatures ranging from 0° C. to physiological temperatures. In one embodiment of the present invention, the neoglycopolymer is water soluble between about 0° C. and about 37° C.

As is known in the art, most synthetic polymers have a distribution of molecular mass and various different averages of the molecular mass are often distinguished, such as the number average molecular mass (Mn) and the weight average molecular mass (M_{w}). The molecular weight of a synthetic polymer is usually defined in terms of its number average molecular mass (Mₙ), which in turn is defined as the sum of nᵢMᵢ divided by the sum of nᵢ, where nᵢ is the number of molecules in the distribution with mass Mᵢ. The neoglycopolymer of the present invention typically has a number average molecular mass (Mn) between 2,000 and 1,000,000. In one embodiment of the present invention, the Mn of the polymer is between about 10,000 and about 80,000. In another embodiment, the Mn of the polymer is between about 40,000 and about 50,000. In a further embodiment, the Mₙ of the polymer is between about 50,000 and about 60,000.

In one embodiment, the neoglycopolymer can actually be a monomeric or oligomeric component thereof. Use of monomeric or oligomeric forms of the neoglycopolymer may result in stiffer matrices because the collagens will be cross-linking with aldehyde moieties that are closer together (i.e., they will both be cross-linked to the same monomer or oligomer).

The neoglycopolymer can be a homopolymer or can be part of a random or block polymer. The neoglycopolymer can be saturated or unsaturated.

In accordance with one embodiment, a copolymer is used to introduce other desired properties or functionalities, including other bioactive groups, as described in more detail below.

Synthesis of the Neoglycopolymer

The neoglycopolymer can be prepared by a ROMP methodology (Mortell, K. H.; Gingras, M.; Kiessling, L. L. J. Am. Chem. Soc. 1994, 116, 12053-4; Fraser, C.; Grubbs, R. H. Macromolecules 1995, 28, 7248-55; Nomura, K.; Schrock, R. R. Macromolecules 1996, 29, 540-5; Nomura, K.; Sakai, I.; Imanishi, Y.; Fujiki, M.; Miyamoto, Y. Macromol. Rapid Commun. 2004, 25, 571-576; Miyamoto, Y.; Fujiki, M.; Nomura, K. J. Polym. Sci., Part A: Polym. Chem. 2004, 42, 4248-4265; Manning, D. D.; Hu, X.; Beck, P.; Kiessling, L. L. J. Am. Chem. Soc. 1997, 119 3161-2; Kanai, M.; Mortell, K. H.; Kiessling, L. L. J. Am. Chem. Soc. 1997, 119, 9931-9932; Strong, L. E.; Kiessling, L. L. J. Am. Chem. Soc. 1999, 121, 6193-6196; and Gordon, E. J.; Gestwicki, J. E.; Strong, L. E.; Kiessling, L. L. Chem. Biol. 2000, 7 9-16). If a saturated polymer is desired, ROMP can be followed by hydrogenation, either as an independent reaction or by using a tandem-ROMP hydrogenation approach (Schnabel, W. Polymer Degradation: Principles and Practical Applications, 1993; Drouin, S. D.; Yap, G. P. A.; Fogg, D. E. Inorg. Chem. 2000, 39, 5412-5414; Drouin, S. D.; Zamanian, F.; Fogg, D. E. Organometallics 2001, 20, 5495-5497; McLain, S. J.; McCord, E. F.; Arthur, S. D.; Hauptman, E.; Feldman, J.; Nugent, W. A.; Johnson, L. K.; Mecking, S.; Brookhart, M. Polym. Mater. Sci. Eng. 1997, 76, 246-247; Bielawski, C. W.; Louie, J.; Grubbs, R. H. J. Am. Chem. Soc. 2000, 122, 12872-12873; and Fogg, D. E.; dos Santos, E. N. Coord. Chem. Rev. 2004, 248, 2365-2379). An example of a tandem ROMP hydrogenation method is also described in U.S. Pat. No. 6,486,263.

The advantage of using a ROMP methodology is that it provides the ability to customize the neoglycopolymer length and allows easy incorporation of other groups of interest by appropriate modification of the substituents on the monomer.

Biopolymer

Biopolymers are naturally-occurring polymers, such as proteins and carbohydrates. The biopolymers useful for incorporation in the biopolymer-based matrix of the present invention contain one or more groups (e.g., a primary amine) that are capable of reacting with the cross-linking moiety of the neoglycopolymer (e.g., aldehyde), or can be derivatised to contain such a group. Examples of suitable biopolymers for use in the present invention include, but are not limited to, collagens (including Types I, II, III, IV and V, human or non-human), recombinant collagens, denatured collagens (or gelatins), fibrin-fibrinogen, elastin, glycoproteins, alginate, chitosan, hyaluronic acid, chondroitin sulphates and glycosaminoglycans (or proteoglycans), as well as cell-interactive glycoproteins such as laminin, fibronectin, tenascin. One skilled in the art will appreciate that some of these biopolymers may need to be derivatised in order to contain a suitable reactive group as indicated above, for example, glucosaminoglycans need to be deacetylated or desulphated in order to possess a primary amine group. Such derivatisation can be achieved using standard techniques and is considered to be within the ordinary skills of a worker in the art. Suitable bio-polymers for use in the invention can be purchased from various commercial sources or can be prepared from natural sources using standard techniques or using standard synthetic or semi-synthetic techniques.

Bioactive Agents

As indicated above, the neoglycopolymer to be included in the biopolymer-based matrix of the present invention contains a plurality of pendant cross-linking groups, for example, cross-linking groups in the form of carbohydrate-masked aldehyde moieties. It will be apparent that sufficient cross-linking of the neoglycopolymer and the biopolymer to achieve a suitably robust matrix can be achieved without reaction of all free cross-linking groups. Excess groups can, therefore, optionally be used to covalently attach desirable bioactive agents to the matrix. Non-limiting examples of bioactive agents that can be incorporated into the matrix by cross-linking include, for example, growth factors, retinoids, enzymes, cell adhesion factors, extracellular matrix glycoproteins (such as laminin, fibronectin, tenascin and the like), hormones, osteogenic factors, cytokines, antibodies, antigens, and other biologically active proteins, certain pharmaceutical compounds, as well as peptides, fragments or motifs derived from biologically active proteins.

In one embodiment of the present invention, the suitable bioactive agents for grafting to the polymer are those which contain primary amino groups, or those which can be readily derivatised so as to contain these groups.

In accordance with a specific embodiment of the present invention, a bioactive group or combination of groups are attached to the neoglycopolymer, for example, as described in Fogg, D. E.; Foucault, H. M., Ring-Opening Metathesis Polymerization. In Comprehensive Organometallic Chemistry III, Hiyama, T., Ed. Elsevier: Oxford, 2006; C. Slugovc. Macromol. Rapid Commun. 25, 2004, 1283; and Kiessling, L. L.; Gestwicki, J. E.; Strong, L. E. Angew. Chem., Int. Ed. 2006, 45, 2348. In one example, copolymers (i.e., a second monomer) are relied on to introduce other covalently bound bioactive groups. This is done by either binding directly to the second monomer or by using a second monomer bearing a functional group that reacts quickly with the desired group for grafting in a post-polymerization reaction.

Cross-Linking the Biopolymer with the Neoglycopolymer

The following discussion refers specifically to collagen as the biopolymer, however, it should be appreciated that collagen is being used as an illustrative example and the present invention is not limited to matrices based on collagen.

Cross-linking of collagen with the neoglycopolymer can be readily achieved by mixing appropriate amounts of each polymer, for example at 0° C., in a suitable solvent. Typically the solvent is an aqueous solvent, such as a salt solution, buffer solution, cell culture medium, or a diluted or modified version thereof. One skilled in the art will appreciate that in order to preserve triple helix structure of collagen and to prevent fibrillogenesis and/or opacification of the matrix, the cross-linking reaction should be conducted in aqueous media with close control of the pH and temperature. The significant levels of amino acids in nutrient media normally used for cell culture can cause side reactions with the cross-linking moieties of the neoglycopolymer, which can result in diversion of these groups from the cross-linking reaction. Use of a medium free of amino acids and other proteinaceous materials can help to prevent these side reactions and, therefore, increase the number of cross-links that form between the neoglycopolymer and the collagen. The cross-linking reaction can be performed in aqueous solution at room or physiological temperatures.

Typically the cross-linking reaction involves the reaction of the aldehyde moieties on the neoglycopolymer (sugar in the ring-opened position) with the primary amines, such as found in lysine or hydroxylysine, on the collagen. In a reducing environment, the resulting cross-link is a secondary amine, as depicted in Scheme I. In one embodiment, the cross-linking reaction can take place in the presence of a reducing agent, such as sodium cyanoborohydride (NaBH₃CN). Alternative suitable reducing agents would be known to those skilled in the art.

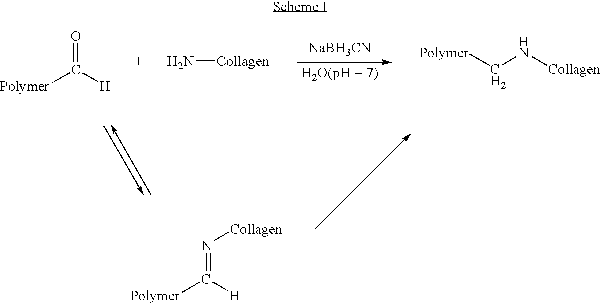

Alternatively, a termination step can be included to react any residual cross-linking groups in the matrix. For example, one or more wash steps in a suitable buffer will remove any unreacted component as well as removing any side products generated during the cross-linking reaction. If necessary, after the cross-linking step, the temperature of the cross-linked polymer suspension can be raised to allow the matrix to form fully.

In accordance with the present invention, the components of the matrix are chemically cross-linked so as to be substantially non-extractable.

One skilled in the art will understand that the amount of each polymer to be included in the matrix will be dependent on the choice of polymers and the intended application for the matrix. In general, using higher initial amounts of each polymer will result in the formation of a more robust matrix due to the lower water content and the presence of a greater amount of cross-linked polymer. Higher quantities of water or aqueous solvent will produce a soft matrix. The matrix can comprise between about 20 and 99% by weight of water or aqueous solvent, between about 0.1 and 30% by weight of neoglycopolymer and between about 0.1 and 50% by weight of collagen. More particularly, the matrix can comprise between about 60 and 99% by weight of water or aqueous solvent, between about 0.1 and 10% by weight of neoglycopolymer and between about 0.1 and 30% by weight of collagen. Even more particularly, the matrix can comprise between about 80 and 98% by weight of water or aqueous solvent, between about 0.5 and 5% by weight of neoglycopolymer and between about 1 and 15% by weight of collagen. The matrix can contain about 94 to 98% by weight of water or aqueous solvent and between about 1-3% by weight of neoglycopolymer and about 1-3% by weight of collagen; or more particularly 95 to 97% by weight of water or aqueous solvent and between about 1-2% by weight of neoglycopolymer and about 2-3% by weight of collagen.

Similarly, the relative amounts of each polymer to be included in the matrix will be dependent on the type of neoglycopolymer and collagen being used and upon the intended application for the matrix. One skilled in the art will appreciate that the relative amounts neoglycopolymer and collagen will influence the final matrix properties in various ways, for example, relatively high quantities of collagen will produce a very stiff matrix. One skilled in the art will appreciate that the relative amounts of each polymer in the final matrix can be described in terms of the weight:weight ratio of the collagen:neoglycopolymer or in terms of equivalents of reactive groups. In accordance with one embodiment of the present invention, the weight per weight (w/w) ratio of collagen:neoglycopolymer is between about 1:0.25 and about 1:5. In one embodiment, the w/w ratio of collagen:neoglycopolymer is between 1:1 and 4:1. In another embodiment, the w/w ratio of collagen:neoglycopolymer is between 5:1 and 1:1.

The ratio of collagen:neoglycopolymer can alternatively be described in terms of molar equivalents of free amine groups in the collagen to aldehyde groups in the neoglycopolymer. In one embodiment, this ratio is between 1:1 and 1:20, and can more particularly be between 1:2 and 1:10.

Uses of the Cross-Linked Biopolymer Matrix

The present invention provides a matrix which is robust, biocompatible and non-cytotoxic and, therefore, suitable for use as a scaffold to allow tissue regeneration in vivo. For example, the matrix can be used for implantation into a patient to replace tissue that has been damaged or removed. A specific example of a use provided by the present invention, is the use of the biopolymer matrix as a cornea substitute, or as a corneal veneer. The matrix can be moulded into an appropriate shape prior to implantation, for example it can be pre-formed to fill the space left by damaged or removed tissue.

In accordance with one embodiment of the present invention, the matrix is pre-formed into an appropriate shape for tissue engineering purposes.

In accordance with one embodiment of the present invention, the matrix is used as an artificial cornea. For this application, the matrix is pre-formed using standard techniques as a full thickness artificial cornea or as a partial thickness matrix suitable for a cornea veneer. In accordance with this embodiment, the matrix is designed to have a high optical transmission and low light scattering.

To gain a better understanding of the invention described herein, the following examples are set forth. It should be understood that these examples are for illustrative purposes only. Therefore, they should not limit the scope of this invention in any way.

### EXAMPLES

General Procedures. All synthetic reactions were carried out at room temperature under N₂ using standard Schlenk or drybox techniques, unless otherwise stated. Solvents were dried using an Anhydrous Engineering solvent purification system and stored over Linde 4 Å molecular sieves in a dry box. Reagents refluxed over and distilled from an appropriate drying agent under a nitrogen atmosphere: triethyl amine and over calcium hydride; methanol over Mg/I₂. RuCl₂(PCy₃)₂(═CHPh) (1) (Schwab, P.; Grubbs, R. H.; Ziller, J. W. J. Am. Chem. Soc. 1996, 118, 100-10), RuCl₂(PCy₃)(IMes)(═CHPh) (2) and RuCl₂(IMes)(Py)₂(═CHPh) (3) (Sanford, M. S.; Love, J. A.; Grubbs, R. H. Organometallics 2001, 20, 5314-5318) catalysts were prepared according to literature procedures. The following materials were purchased from Aldrich and used without purification: 1,2:3,4-di-O-isopropylidene-α-D-galactopyranose, fumaryl chloride, cyclopentadiene, Na₂HPO₄, KH₂PO₄, NaBH₃CN. Hydrogen (UHP Grade) was purchased from Praxair and used without purification. Deuterated solvents were obtained from Cambridge Isotope Laboratories Ltd. Ethyl vinyl ether was degassed by consecutive freeze/pump/thaw cycles. Bis(1,2:3,4-di-O-isopropylidene-D-galactopyranos-6-O-yl) 5-norbornene-2,3-dicarboxylate (NBE-digal) was prepared according to literature procedures (Nomura, K.; Schrock, R. R. Macromolecules 1996, 29, 540-5). All gel matrices described in the Examples set out below used sterile collagen I, such as atelocollagen (bovine, porcine or recombinant), which can be prepared in the laboratory or more conveniently is available commercially (for example, from Becton Dickinson, and from Fibrogen). Collagen solutions were adjusted to physiological conditions, i.e. saline ionic strength and pH 7.2-7.4, through the use of aqueous sodium hydroxide in the presence of PBS. 10% collagen solutions were made prior to the cross-linking reaction. A PBS (phosphate-buffered saline, pH=7) was prepared to yield concentrations of KCl 0.2 g·L⁻¹; NaCl 8 g·L⁻¹; Na₂HPO₄ 1.15 g·L⁻¹; and KH₂PO₄ 0.2 g·L⁻¹H NMR (300 or 500 MHz) and ¹³C NMR (75 MHz) spectra were recorded on a Bruker Avance-300 or Bruker AMX-500 spectrometer. GPC data were obtained using CH₂Cl₂ as eluent (flow rate 1.0 ml/min; samples 1-2 mg/ml) on a Wyatt DAWN light-scattering instrument equipped with an Optilab DSP refractometer, an HPLC system with a Waters model 515 pump, Rheodyne model 7725i injector with 200 μL injection loop, and Waters Styragel HR3 and HR4 columns in series. Infrared spectra were recorded on a Bomem MB100, Bomem Michelson M129, or Shimazu FTIR-8400S IR spectrometer. Samples were run as KBr pellets (20 mg polymer/200 mg KBr) prepared using a RIIC (Research Industrial Instruments Company) ring press. For temperature-controlled experiments, IR samples were placed in a cylindrical sample holder and heated with a Lambda model LP-521-FM regulated power supply. Hydrogel tensile strengths were measured on a Model 1123 Instron Tensile Testing machine. Refractive indices were measured on an Abbe refractometer (Bellingham and Stanley, UK) at 21° C., illuminated with the sodium D-line.

### Example 1

### Polymerization of Monomeric Starting Material (NBE-Digal) Using ROMP to Form Unsaturated Polymeric Product

Bis(1,2:3,4-di-O-isopropylidene-D-galactopyranos-6-O-yl) 5-norbornene-2,3-dicarboxylate (NBE-digal) was polymerized using different Ru-based catalysts shown below: 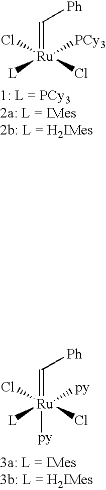
2a and 3a represent the catalyst during the ROMP (i.e., the polymerization reaction). 2b and 3b are the same catalyst as 2a and 3a, respectively, except that they represent the catalyst as it would be during the hydrogenation reaction. Hydrogen gas (H₂) would be activated by the catalyst during the hydrogenation, through coordination of the H₂ to the catalyst as set out in Scheme II below:

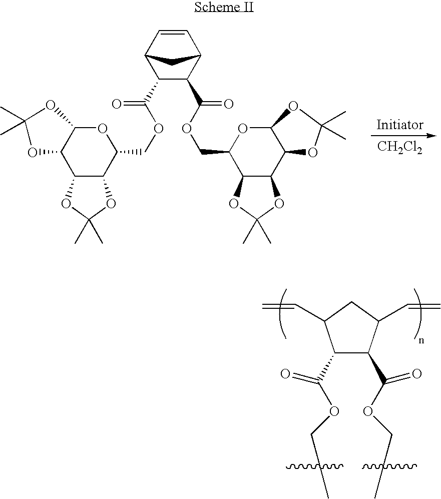

In a representative procedure, a solution of Bis(1,2:3,4-di-O-isopropylidene-D-galactopyranos-6-O-yl) 5-norbornene-2,3-dicarboxylate (NBE-digal) (121 mg, 0.175 mmol) in 1 ml CH₂Cl₂ was added in one portion to a rapidly stirred solution of RuCl₂(PCy₃)₂(═CHPh) (1) (3 mg, 0.0036 mmol) in 2 ml CH₂Cl₂. Conversions were determined by monitoring the decrease in the monomer olefinic resonances (6.23-6.11 ppm) relative to tetrakis(trimethylsilyl)silane (TMSS) internal standard (Demel, S.; Schoefberger, W.; Slugovc, C.; Stelzer, F. J. Mol. Catal. A 2003, 200, 11-19), and relative to the carbohydrate methine resonance at 4.59 ppm. After complete conversion (¹H NMR) the polymerization was quenched by adding ethyl vinyl ether, and stirred for 20 minutes. The solvent was stripped, the resulting solid dissolved in the minimum amount of CH₂Cl₂, and added dropwise to vigorously stirred cold methanol (˜100 ml) to afford a pink precipitate. The polymer was collected by centrifugation, purified by column chromatography (70% ethyl acetate in hexanes) and dried in vacuo. NMR data agreed with values previously reported (Nomura, K.; Schrock, R. R. Macromolecules 1996, 29, 540-5).

Mn

| Cat. | t/h | Calc. | Found | PDI | % Conv. |
|---|---|---|---|---|---|
| 1 | 48 | 33400 | 34700 | 1.08 | 80-90 |
| 2 | 3 | 33400 | 14800 | 1.23 | 100 |
| 3 | <3 | 33400 | 36050 | 1.03 | 100 |

Cat. = catalyst,
t/h = time (hours),
Mn = number-average molecular weight,
PDI = polydispersity,
% Conv. = percent conversion

### Example 2

### Polymerization of Monomeric Starting Material using Tandem ROMP-Hydrogenation to Form Saturated Polymer

The polymerization was performed using the same initiators as described above according to Scheme III:

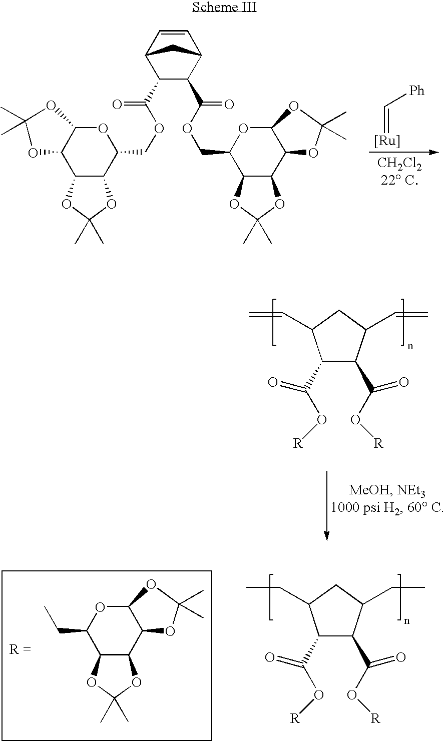

In a representative procedure, a solution of NBE-digal (121 mg, 0.175 mmol) in 1 ml CH₂Cl₂ was added in one portion to a rapidly-stirred solution of 1 (3 mg, 0.0036 mmol) in 2 ml CH₂Cl₂. Once reaction was complete, the solution was diluted with CH₂Cl₂, then MeOH, and NEt₃ was added (5 μl, 0.036 mmol). The solution was then purged with H₂ in a glass-lined autoclave, pressurized to 1000 psi and stirred at 60° C. For establishment of time profiles, samples were removed at intervals for ¹H NMR analysis, monitoring decreases in the integrated intensity of the olefinic peaks (5.60-5.30) relative to the carbohydrate methine signal at 4.59 ppm. Following complete hydrogenation, the solvent was stripped off, and the resulting solid dissolved in the minimum amount of CH₂Cl₂, then added dropwise to vigorously stirred cold methanol (˜100 ml). The polymer precipitate was collected by centrifugation, purified by column chromatography (70% ethyl acetate in hexanes) and dried in vacuo. δ ¹H NMR (CDCl₃, 500.1 MHz, 298K) δ 5.48 (br s, 2H, anomeric sugar proton), 4.58 (br s, 2H, sugar CHCH₂), 4.28-4.13 (br m, 8H, sugar group protons+CH₂), 3.98 (br m, 2H, sugar group protons); 3.13 (br m, 1H, 5-membered ring proton), 2.78 (br m, 1H, 5-membered ring proton), 2.2-1.8 (br m, 2H, 5-membered ring protons), 1.8-1.5 (br m, 2H, 5-membered ring CH₂); 1.5-1.2 (br m, 2H, protons of polymer backbone); 1.46 (s, 6H, acetal CH₃), 1.41 (s, 6H, acetal CH₃), 1.30 (br s, 12H, acetal CH₃). ¹³C{¹H} NMR (CDCl₃, 75 MHz, 298K) δ175.3, 174.5 (carbonyl), 109.8, 109.0 (isopropylidene), 96.6 (anomeric sugar CH), 71.3, 71.1, 70.8, 66.2, 63.8 (sugar group CH+CH₂), 53.1, 51.4, 44.8, 44.1, 38.3 (5-membered ring CH), 30.0 (methylene protons of polymer backbone), 26.4, 25.4, 24.9 (isopropylidene). IR (Nujol): ν(CO) 1730 cm⁻¹.

the results for the hydrogenation component of the ROMP-hydrogenation reaction are as follows:

Mn

| Cat. | t/h | Calc. | Found | PDI | % Conv. |
|---|---|---|---|---|---|
| 1 | 2.5 | 33400 | 30140 | 1.12 | 100 |
| 2 | 2.5 | 33400 | 121000 | 1.32 | 100 |
| 3 | 24 | / | / | / | 38 |

Cat. = catalyst,
t/h = time (hours),
Mn = number-average molecular weight,
PDI = polydispersity,
% Conv. = percent conversion

### Example 3

### Homopolymer Deprotection

Prior to cross-linking the neoglycopolymer was deprotected according to Scheme IV:

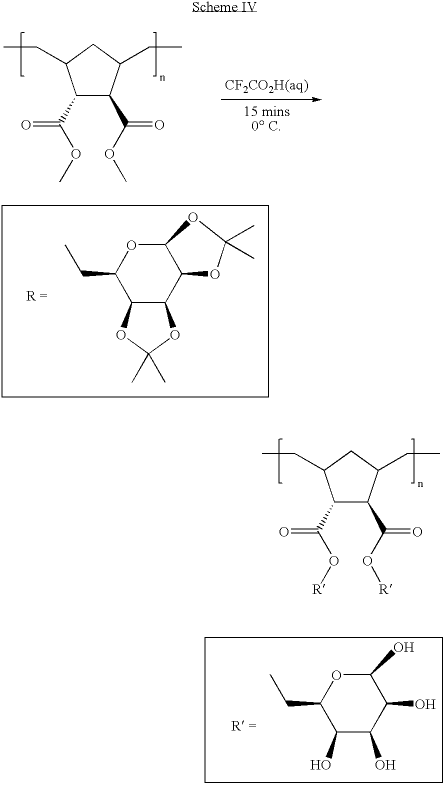

In a representative procedure, trifluoroacetic acid in H₂O (9/1, v/v, 1 ml) was added to the acetal-protected polymer (100 mg) and the mixture was stirred for 20 min until the suspension became fully soluble. The solution was added dropwise to 50 ml THF at 0° C., with vigorous stirring. The white precipitate was filtered off, washed in THF (40 ml), Et₂O (2×40 ml) and hexanes (2×40 ml), and dried in vacuo. ¹H NMR (D₂O, 300.1 MHz, 298K) δ 5.2 (br m, 2H, anomeric CH), 4.9-4.6 (br m, OH), 4.6-3.6 (br m, CH, CH₂), 3.4-2.0 (br m, furanose CH), 1.3-1.0 (br m).

### Example 4

### Collagen Cross-Linking

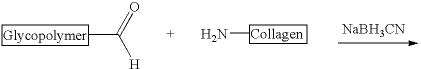

In a representative procedure, aqueous collagen solution (0.5 ml, 10% w/w) was taken into a bubble-free syringe mixing system in an ice-water bath (Li, F.; Griffith, M.; Li, Z.; Tanodekaew, S.; Sheardown, H.; Hakim, M.; Carlsson, D. J. Biomaterials 2005, 26, 3093-3104). Homopolymer (0.28 ml, 20% w/v in PBS solution) was added using a second syringe, and mixed thoroughly with the collagen solution, following which the pH of the mixture was adjusted to 7 by using 1.0 M NaOH. NaBH₃CN (0.026 ml, 10% in PBS) solution was added and mixed thoroughly. The homogenous solution was then dispensed into contact lens moulds (500 μm spacing) and cured at 100% humidity, first at room temperature (5 days) and then at 37° C. (24 hours). The cross-linked, cornea-shaped hydrogel samples were removed from the moulds, washed in PBS and stored in PBS containing 1% chloroform to maintain sterility.

Tensile Testing of Hydrogels. In a representative procedure, tensile strength was determined using the suture pull-out method (Li, F.; Griffith, M.; Li, Z.; Tanodekaew, S.; Sheardown, H.; Hakim, M.; Carlsson, D. J. Biomaterials 2005, 26, 3093-3104). The fully hydrated, moulded cornea implant sized gels (500 μm thickness, 12 mm diameter) were suspended between two diametrically opposed nylon 10/0 sutures (33 μm diameter monofilaments) penetrating through the gel at 2 mm in from the edge of the hydrogel. Paired free ends of each suture were clamped in one of the two micro-clamps on an Instron Tensile Testing Machine and each sample drawn to break at a rate of 10 mm/min.

Physical properties of collagen-homopolymer cross-linked hydrogel

| Collagen-polymer ratio | Refractive | Stress at Break Point |
|---|---|---|
| (w/w) | Index | (g) |
| 0:1 | opaque | N/A |
| 1:1 | 1.3485 | 6.16 |
| 2:1 | 1.3452 | 6.35 |
| 4:1 | 1.3432 | 7.26 |
| 1.5:1^{b} | 1.3427 | 5.40 |

### Example 5

### Neoglycopolymer-Cross-Linked Matrix

Collagen, the principal structural element of the extracellular matrix (ECM), has been extensively applied to tissue engineering fields such as in skin substitutes, vascular grafts, cartilage scaffold, bone implants and corneal substitutes due to a role in favoring the attachment, migration and differentiation of cells. Its effectiveness is adversely affected, however, by low mechanical strength and rapid enzymatic biodegradation. Chemical cross-linking methods have proven to be effective in improving the mechanical properties and resistance of collagen to enzyme degradation. Among the cross-linkers currently used, glutaraldehyde and water-soluble carbodiimide (WSC) are the most common ones. But the notorious cytotoxicity of glutaraldehyde is cause for concern. WSC, a zero-length cross-linking agent, can provide both intrahelical and interhelical cross-links between adjacent collagen microfibrils without integrating foreign molecules into the network. Thus, WSC is regarded as an effective and benign cross-linking agent. Of particular interest is the use of WSC-cross-linked collagen matrices as corneal substitutes, which are clear, robust, suturable and biocompatible. Recently, genipin, a naturally occurring product extracted from gardenia fruits has gained increasing attention as a cross-linking agent of protein or polysaccharide-based tissue engineering scaffold. It is shown to be much less toxic than glutaraldehyde. However, the dark blue or brown color intrinsic to genipin makes it unsuitable for corneal substitute cross-linking use.

Increases in tissue stiffness associated with aging or diabetes are known to result from glycation, a process involving nonenzymatic cross-linking of amine groups of collagen and other ECM proteins by reducing sugars. The key initial step involves formation of Schiff base products via condensation of aldehyde groups of the ring-opened sugars with amine groups present in lysine and hydroxylysine residues. While carbohydrate molecules, including glucose and ribose, have previously been used to cross-link collagen, the present invention relates to use of carbohydrate-functionalized polymers with tunable functionality and dimension, as a means for improving mechanical properties while minimizing reduction in transparency.

As described in more detail herein, ROMP of cyclic monomers offers powerful synthetic methodologies for molecular-level design of macromolecular materials, enabling specification of chain lengths, microstructure, and the nature and density of pendant groups. ROMP techniques have been used previously for construction of carbohydrate-functionalized polymers: of particular interest in the present context, Kiessling and coworkers have demonstrated that ROMP neoglycopolymers can participate in interactions with cell surfaces. Reduction of the ROMP polymers is important in order to retain control of polymer microstructure, and while this is most commonly effected in a post-polymerization procedure (by, for example, diimide reduction), as demonstrated herein, tandem processes of Ru-catalyzed ROMP and hydrogenation (Scheme V) can provide efficient routes to saturated polyolefins. Here we report the application of tandem, Ru-catalyzed ROMP-hydrogenation methodologies to construction of saturated neoglycopolymers that prove effective crosslinking agents for construction of collagen-based cornea hydrogels.

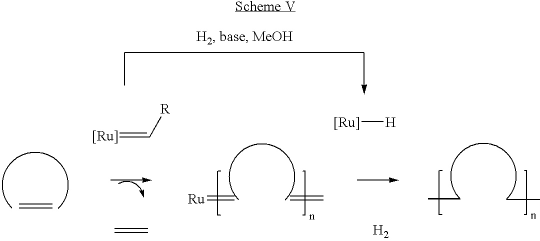

Materials and method

Materials: Porcine type I atelocollagen was purchased from Nippon Ham, Japan. Neoglycopolymers (n=50, Mn=25300; n=120, Mn=61567; n=170, Mn=85120) were prepared according to the techniques outline above. Sodium cyanoborohydride was supplied by Aldrich. All other reagents were of analytical grade and used as received.

Preparation of Hydrogels: 0.2 ml of 13.7 wt % collagen solution and 0.1 ml of PBS were mixed in syringes in ice-water bath. After a homogenous solution was formed, 0.2 ml of neoglycopolymer (6.85 wt % in PBS) was injected into the mixture with a ratio to collagen 2:1 (w/w). The pH of solution was adjusted up to 8 by injecting 30 μl of NaOH. Then 20 μl of NaBH₃CN solution (1 mg/μl in PBS) was added through another microsyringe. The mixture was cast into a glass mould and left at room temperature with 100% humidity for 48 h. Then the moulds were transferred into an incubator for post-curing at 37° C. for 1 day. Analogously, The hydrogels with weight ratios of neoglycopolymer to collagen 1:1 and 1:3 and 1:4 were prepared.

Herein, the codes Coll-neo50-1, Coll-neo50-2, Coll-neo50-3 and Coll-neo50-4 denote the gels cross-linked by neoglycopolymer (n=50) with weight ratios of collagen/neoglycopolymer=1/1, 2/1, 3/1 and 4/1, respectively.

In the same way, hydrogels cross-linked by neoglycopolymers (n=120, n=170) were also prepared at ratios of collagen/neoglycopolymer=1/1, 2/1, 3/1, and 4/1, which were coded as Coll-neol20-1, Coll-neol20-2, Coll-neol20-3, Coll-neol20-4 and Coll-neo 170-1, Coll-neo 170-2, Coll-neo 170-3 and Coll-neo 170-4, respectively.

Solid-state ¹³C NMR: Coll-neo50-2 hydrogel was dried and cut into small fragments. NMR measurement was performed on a Bruker AVANCE 500 MHz spectrometer. The samples were packed into 4 mm zirconia rotors and spun at 14 kHz.

Optical property measurements: Refractive indices (RIs) of fully hydrated hydrogels were recorded on a VEE GEE refractometer at 21° C. with bromonaphthalene as the calibration agent. PBS equilibrated thin and flat hydrogels were used for the RI measurements.

Transmission measurement of cornea-shaped hydrogels was made, both for white light (quartz-halogen lamp source) and over narrow spectral regions (Δν_{1/2} of 40 nm centered at 450, 500, 550, 600 and 650 nm), on a custom-built instrument.

Mechanical property measurement: The tensile strength, elongation at break, and elastic moduli of the hydrogels were determined on an Instron's electromechanical universal tester (Model 3340) equipped with Series IX/S software. Flat hydrogels, 0.50 mm thick, were equilibrated in PBS and cut into 12 mm×5 mm rectangular sheets. To enhance the gripping of the clips and prevent damage of the specimen from clipping, two ends of each specimen were glued to a mounting tape using tissue adhesive, Dermabond™ (Ethicon Inc.). The actual gauge length of each specimen is 5 mm for testing. Five specimens were measured for each hydrogel formulation. The crosshead speed was at 10 mm min⁻¹. Robustness for transplantation was also examined by looking at the suturability of the hydrogels. Suturability of cornea-shaped implants was evaluated by determining their ability to tolerate placements of 16 sutures without shearing, using polyamide, black monofilaments (Ethicon, 10-0, 33 μm, black sutures).

Equilibrated water content: After removal from the moulds, hydrogels were immersed in PBS for 7 days at 4° C. and 6 h at room temperature. The hydrogels were taken out of the PBS and the surface was gently blotted with filter paper, after which the hydrogels were weighed on a microbalance to record the wet weight of the samples. These hydrogels of known weight were then dried at room temperature under vacuum until a constant weight was attained. The total equilibrated water content of hydrogels (Wₜ) was calculated according to the following equation:
Wₜ=(W−W₀)/W×100%
where W₀ and W denote weights of dried and PBS equilibrated samples, respectively.

Differential scanning calorimetry (DSC) analysis: The thermal properties of hydrogels were measured on a Perkin-Elmer DSC-2C differential scanning calorimeter. Heating scans were recorded in the range of 20 to 70° C. at a scan rate of 10° C. min⁻¹. PBS-equilibrated RHC hydrogels, with weights ranging from 5 to 10 mg, were surface-dried with filter paper and hermetically sealed in an aluminum pan to prevent water evaporation. For comparison, a 13.7% collagen solution was also measured in the same fashion. PBS was used as a blank reference.

In vitro biocompatibility and performance: Immortalized corneal epithelial cells (HCEC) were used to evaluate epithelial coverage, using a slight modification of a previously described method (Li, F.; Carlsson, D.; Lohmann, C.; Suuronen, E.; Vascotto, S.; Kobuch, K.; Sheardown, H.; Munger, R.; Nakamura, M.; Griffith, M. Proc. Nat. Acad. Sci. USA 2003, 100, 15346-15351). Briefly, HCECs were seeded on top of 1.5 cm² hydrogel pieces, supplemented with a serum-free medium containing epidermal growth factor (Keratinocyte Serum-Free Medium (KSFM; Life Technologies, Burlington, Canada)) and grown until confluent. The medium was then switched to a serum-containing modified SHEM medium for 2 days, followed by maintenance at an air/liquid interface. At 2 weeks, constructs were fixed in 4% paraformaldehyde (PFA) in 0.1 M PBS and processed for routine haematoxylin and eosin (H&E) staining. Time to confluence was compared to plasma-treated, tissue culture plastic controls, and the ability of hydrogels to support epithelial stratification was evaluated.

To determine the ability of the hydrogels to support nerve surface growth, dorsal root ganglia (DRG) from chick embryos (E 8.0) were dipped into collagen matrix as an adhesive, and adhered to the surface of washed gel pieces. Neurite growth was observed for up to a total of 5-6 days, after which the gels were fixed in 4% paraformaldehyde in 0.1 M PBS, pH 7.2-7.4 and stained for the presence of neurofilament using mouse anti-NF200 antibody overnight at 4° C. Neurofilament was visualized the following day using donkey antimouse-Cy2 secondary antibody. Whole-mounts were imaged using a Zeiss Axiovert microscope.

In vitro biodegradation: 50-80 mg of hydrated hydrogels were placed in vials containing 5 ml 0.1 M PBS (pH 7.4), followed by addition of 60 ul of 1 mg/mL of collagenase (Clostridium histolyticum, EC 3.4.24.3, Sigma Chemical Co.). Then the vials were incubated in an oven at 37° C., and at different time intervals, the gels were taken out for weighing with surface water wiping off. Time course of residual mass of hydrogels was tracked based on the initial swollen weight.

Rat subcutaneous implantation: Incision was made on the skin at the back of the rat. The neoglycopolymer-cross-linked collagen hydrogels were inserted under the skin and the skin incision was closed.

Results and Discussion

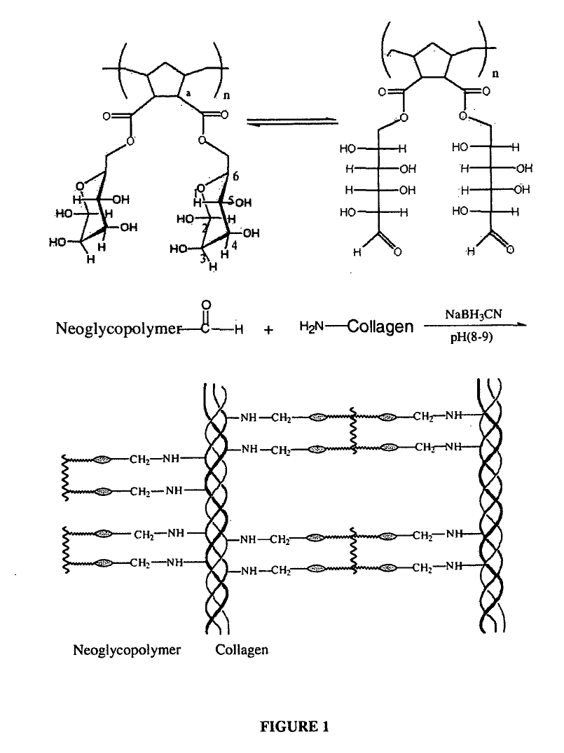

Formation of neoglycopolymer-crosslinked hydrogels: Under basic conditions, the dangling glucose units in the neoglycopolymer tend to open rings to generate free aldehyde groups, which are able to react with amines of lysine and hydroxylysine in collagen. The reaction scheme is presented in FIG. 1. The unstable imine linkages were reduced by NaBH₃CN.

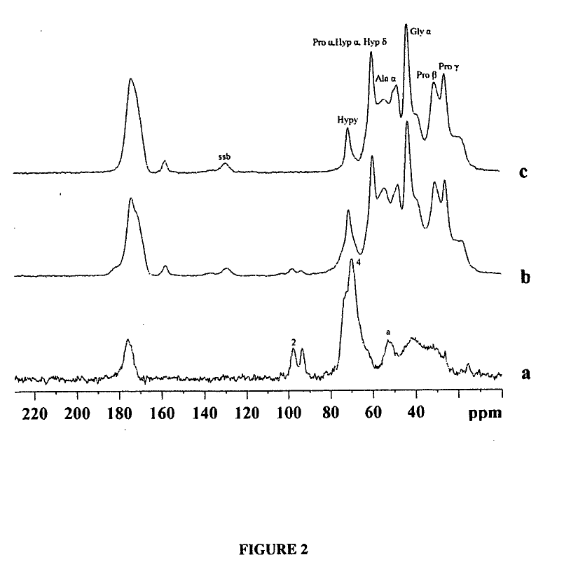

FIG. 2 depicts the solid-state ¹³C NMR spectra of neoglycopolymer, Coll-neo50-2 and collagen. It is evident that the typical feature bands are present in Porcine type I atelocollagen (Daniel Huster, Jurgen Schiller and Klaus Arnold, Magnetic Resonance in Medicine (2002), 48:624-632). Compared with pure collagen, twin peaks around 96 ppm attributed to C2 appear in the spectra for both the neoglycopolymer and the Coll-neo50-2, indicating the occurrence of cross-linking.

Optical properties: In designing cornea substitutes, an ideal optical property is the successful use in actual clinical application. Table 1 lists the optical transmission data determined at varied wavelengths of hydrogels crosslinked with different chain lengths of neoglycopolymer at various ratios. For all three crosslinkers, except for 1:1 collagen/neoglycopolymer ratio, the light transmission of hydrogels are transparent and superior to that of human cornea at white light length.

TABLE 1
Optical Transmission

| | | | | | | |
|---|---|---|---|---|---|---|
| Wavelength(nm) | White | 450 | 500 | 550 | 600 | 650 |

Average Transmission (%)

| | | | | | | |
|---|---|---|---|---|---|---|
| Coll-neo50-1 | 75.3 ± 0.5 | 66.7 ± 0.5 | 69.8 ± 0.6 | 73.0 ± 0.7 | 76.4 ± 0.7 | 78.7 ± 0.5 |
| Coll-neo50-2 | 89.7 ± 2.4 | 86.0 ± 1.4 | 83.2 ± 0.9 | 82.8 ± 2.0 | 83.3 ± 2.6 | 83.9 ± 2.7 |
| Coll-neo50-3 | 88.2 ± 4.7 | 59.8 ± 5.4 | 73.4 ± 3.6 | 79.5 ± 1.6 | 81.4 ± 4.7 | 84.8 ± 3.9 |
| Coll-neo50-4 | 83.7 ± 1.1 | 72.9 ± 5.8 | 78.2 ± 4.0 | 80.6 ± 3.5 | 82.5 ± 3.1 | 83.7 ± 2.8 |
| Coll-neo120-1 | 71.8 ± 8.0 | 55.7 ± 1.7 | 64.8 ± 1.5 | 69.0 ± 0.8 | 73.5 ± 0.8 | 76.9 ± 0.2 |
| Coll-neo120-2 | 89.8 ± 0.9 | 65.7 ± 5.0 | 80.8 ± 6.8 | 85.2 ± 4.6 | 87.4 ± 4.0 | 89.7 ± 4.5 |
| Coll-neo120-3 | 83.2 ± 2.3 | 49.9 ± 3.4 | 66.9 ± 4.5 | 75.2 ± 5.5 | 78.5 ± 5.7 | 81.1 ± 5.9 |
| Coll-neo120-4 | 87.0 ± 2.9 | 63.9 ± 6.3 | 79.0 ± 3.9 | 84.3 ± 3.7 | 86.2 ± 3.0 | 88.0 ± 2.7 |
| Coll-neo170-1 | 73.5 ± 2.1 | 37.0 ± 0.7 | 53.7 ± 1.8 | 62.1 ± 2.8 | 66.7 ± 3.5 | 71.0 ± 4.0 |
| Coll-neo170-2 | 86.7 ± 1.2 | 57.2 ± 1.2 | 74.2 ± 0.5 | 80.9 ± 0.2 | 83.7 ± 0.7 | 86.2 ± 1.3 |
| Coll-neo170-3 | 83.1 ± 2.4 | 44.0 ± 1.9 | 63.9 ± 0.9 | 73.6 ± 0.4 | 78.2 ± 0.2 | 84.7 ± 2.5 |
| Coll-neo170-4 | 81.0 ± 0.7 | 48.2 ± 2.9 | 66.4 ± 2.5 | 74.9 ± 1.6 | 79.3 ± 1.3 | 83.9 ± 0.1 |

The refractive indices of hydrogels ranged from 1.3427 to 1.3478 (Table 2), which is slightly lower than that of human corneal stroma at 1.373-1.380. The effect of cross-linker contents on the refractive indices of hydrogels was negligible.

TABLE 2
RI and equilibrated water contents of Collagen Hydrogels

| Sample | RI | Water content(%) |
|---|---|---|
| Coll-neo50-1 | 1.3461 ± 0.0001 | 91.17 ± 0.85 |
| Coll-neo50-2 | 1.3460 ± 0.0005 | 91.85 ± 0.66 |
| Coll-neo50-3 | 1.3432 ± 0.0007 | 93.48 ± 0.65 |
| Coll-neo50-4 | 1.3427 ± 0.009 | 91.97 ± 0.49 |
| Coll-neo120-1 | 1.3454 ± 0.0002 | 92.25 ± 0.36 |
| Coll-neo120-2 | 1.3446 ± 0.0001 | 94.03 ± 0.88 |
| Coll-neo120-3 | 1.3441 ± 0.0001 | 91.12 ± 0.51 |
| Coll-neo120-4 | 1.3478 ± 0.0004 | 90.60 ± 0.12 |
| Coll-neo170-1 | 1.3449 ± 0.0003 | 92.11 ± 0.73 |
| Coll-neo170-2 | 1.3443 ± 0.0002 | 91.09 ± 0.82 |
| Coll-neo170-3 | 1.3438 ± 0.0003 | 92.33 ± 0.75 |
| Coll-neo170-4 | 1.3460 ± 0.0007 | 93.69 ± 0.67 |

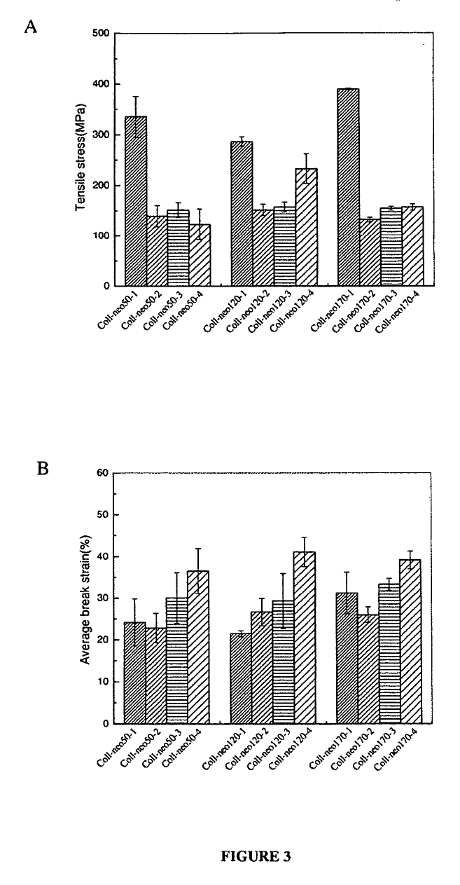

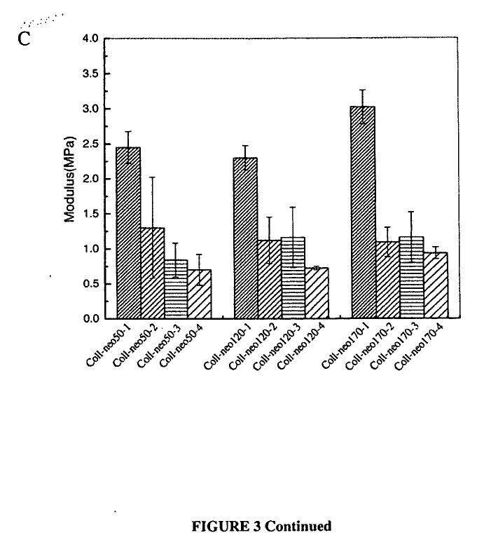

Mechanical properties: From the point of view of mechanics, a potential corneal substitute must be strong enough to withstand the manipulation of suture thread and needle with more than 90% water. The water contents of all the hydrogels prepared in this work are over 90% (Table 2). The tensile strength, break strain and elastic moduli of hydrogels with various neoglycopolymer ratios were measured. As shown in FIG. 3, the average tensile stress of hydrogels is in the range of 87-440 KPa; the breaking strain is 20-40%, and the moduli are 2.18-2.99 MPa.

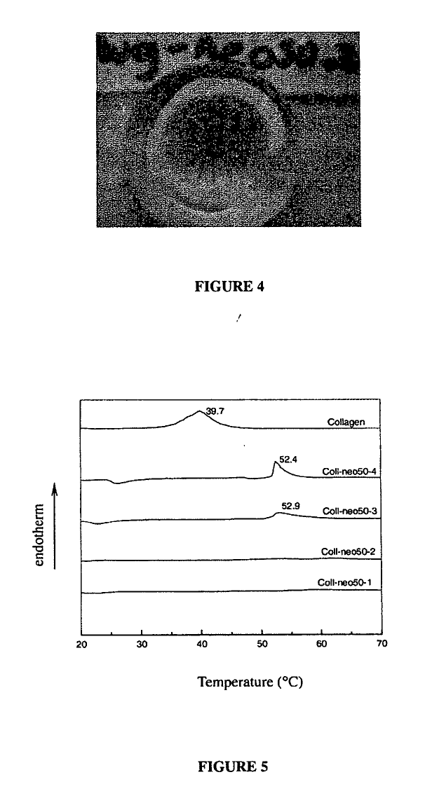

Since Coll-neo50-2 displayed the best transmission, it was chosen for suturability evaluation (FIG. 4). No tearing or microshearing of the suture points were observed following suturing of this corneal substitute onto a donor human corneal rim using 16 sutures. These data indicate that the Coll-neo50-2 exhibits mechanical properties suitable for use as a corneal substitute.

DSC and in vitro degradation: Corneal substitutes fabricated from collagen hydrogels should be stable enough to withstand denaturation of collagen helices in vivo, which occurs when temperature fluctuates. FIG. 5 demonstrates the DSC heating thermograms of collagen solution and Coll-neo50 hydrogels at different neoglycopolymer ratios. The thermodynamic data are collected in Table 3.

TABLE 3
Thermodynamic data of hydrogels from DSC

| | Collagen | Coll- | Coll- | Coll- | Coll- |
|---|---|---|---|---|---|
| Samples | solution | neo50-4 | neo50-3 | neo50-2 | neo50-1 |
| T_{d}(° C.) | 39.7 | 52.4 | 52.9 | —* | — |
| ΔH_{d}(J/g | 67.3 | 62.1 | 40.9 | — | — |
| xerogel) | | | | | |

*cannot be detected

For the collagen solution, a transition peak appears at approximately 39.7° C., i.e., denaturation temperature (Td), and the enthalpy (AHd) is about 67.3 J/g xerogel, implying a typical helix-coil transition. A notable variation trend is that with neoglycopolymer cross-linking, the peak shifts toward high temperature at collagen/neoglycopolymer ratios, 4/1 and 3/1, and disappears at higher neoglycopolymer contents, 2/1 and 1/1. In contrast, the enthalpy of hydrogel decreases with the increase of neoglycopolymer contents (Table 2), suggesting the helical structure is considerably stabilized by polymer cross-linking. DSC thermograms were also obtained for collagen hydrogels cross-linked with WSC at molar ratios of WSC to amine of collagen, 1/1 and 3/1. At 1/1 ratio, the peak is located at 50.1° C.; at 3/1, no peak was observed. This further verifies that neoglycopolymer cross-linking is effective to enhance the thermostability of hydrogels.

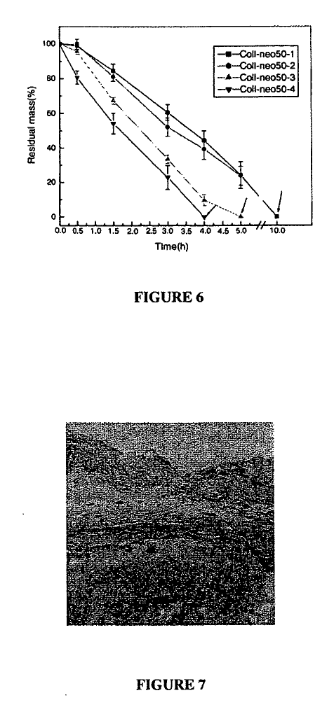

Apart from thermostability, a potential corneal substitute should be biodegradable so as to allow tissue remodelling. FIG. 6 depicts in vitro degradation kinetics of neoglycopolymer-cross-linked hydrogels. As shown in FIG. 6, Coll-neo50-4, Coll-neo50-3, Coll-neo50-2 and Coll-neo50-1 were completely degraded within 4, 5 and 10 h, respectively, indicating higher neoglycopolymer ratios lead to the enhanced biostability. However, the degradation is fast due to the high collagenase concentration.

Rat subcutaneous implantation: After 60 days implantation, it was found that the hydrogel was very biocompatible without any inflammation.

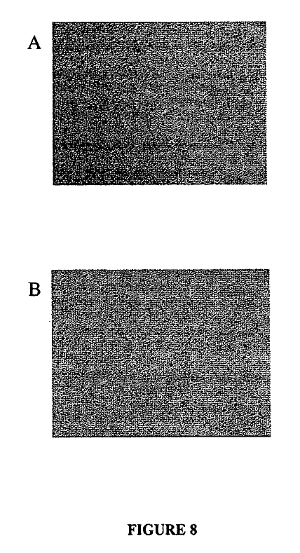

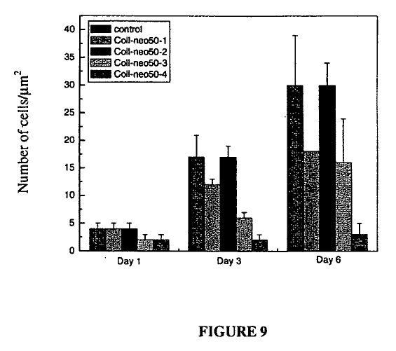

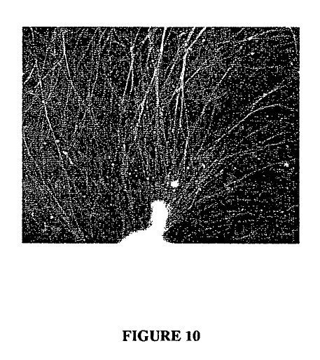

Human corneal epithelial and nerve growth: FIG. 8 depicts pictures of human corneal epithelial growth on the surface of Coll-neo50-2 hydrogel and plate cultured at 6 days. FIG. 9 summarizes the quantitative analysis of cell proliferation on the surface of gels cross-linked with various ratios of neoglycopolymer (n=50). Coll-neo50-2 gel demonstrates good cell affinity. At day 6, the number of cells on the surface of this hydrogel is augmented and equivalent to the control, indicating that Coll-neo50-2 supports attachment and proliferation of corneal epithelial cells. FIG. 10 is a photograph demonstrating nerve growth on the surface of Coll-neo50-2.

### REFERENCES

(1) Lee, K. Y.; Mooney, D. J. Chem. Rev. 2001, 101, 1869-1879.
(2) Langer, R.; Vacanti, J. P. Science 1993, 260, 920-926.
(3) Atala, A.; Mooney, D. J.; Vacanti, J. P.; Langer, R. S., Ed. Synthetic Biodegradable Polymer Scaffolds. Birkhauser: Boston, 1997.
(4) Shoichet, M. S.; Hubell, J. A., Ed. Polymers for Tissue Engineering. VSP: Utrecht, Netherlands, 1998.
(5) Griffith, M.; Osborne, R.; Munger, R.; Xiong, X.; Doillon, C. J.; Laycock, N. L. C.; Hakim, M.; Song, Y.; Watsky, M. A. Science 1999, 286, 2169-2172.
(6) Weinberg, C. B.; Bell, E. Science 1986, 231, 397-400.
(7) Bell, E.; Ehrlich, H. P.; Buttle, D. J.; Nakatsuji, T. Science 1981, 211, 1052.
(8) Girton, T. S.; Oegema, T. R.; Tranquillo, R. T. J. Biomed. Mat. Res. 1999, 46, 87-92.
(9) Hoffman, A. S. Adv. Drug. Deliv. Rev. 2002, 43, 3-12.
(10) Rault, I.; Frei, V.; Herbage, D.; Abdul-Malak, N.; Huc, A. J. Mater. Sci. Mater. Med. 1996, 7, 215-21.
(11) Weadock, K.; Olson, R. M.; Silver, F. H. Biomat. Med. Dev. Art. Org. 1984 11 293-318.
(12) Shimmura, S.; Doillon, C., J.; Griffith, M.; Nakamura, M.; Gagnon, E.; Usui, A.; Shinozaki, N.; Tsubota, K. Cornea 2003, 22, S81-8.
(13) Li, F.; Carlsson, D.; Lohmann, C.; Suuronen, E.; Vascotto, S.; Kobuch, K.; Sheardown, H.; Munger, R.; Nakamura, M.; Griffith, M. Proc. Nat. Acad. Sci. USA 2003, 100, 15346-15351.
(14) Li, F.; Griffith, M.; Li, Z.; Tanodekaew, S.; Sheardown, H.; Hakim, M.; Carlsson, D. J. Biomaterials 2005, 26, 3093-3104.
(15) Jager, M. J. Eye 1995, 9, 241.
(16) Nimni, M. E. J. Biomed. Mat. Res. 1987, 21, 741-771.
(17) Monnier, V. M.; Sell, D. R.; Abdul-Karim, F. W.; Emancipator, S. N. Diabetes 1988, 37, 867-72.
(18) Monnier, V. M.; Kohn, R. R.; Cerami, A. Proc. Nat. Acad. Sci. USA 1984, 81, 583-587.
(19) King, G. L.; Brownlee, M. Endocrinol. Metab. Clin. North Am. 1996, 25, 255-270.
(20) Brownlee, M. Diabetes 1992, 41, 57-60.
(21) Ohan, M. P.; Weadock, K. S.; Dunn, M. G. J. Biomed. Mat. Res. 2002, 60, 384-391.
(22) Mortell, K. H.; Gingras, M.; Kiessling, L. L. J. Am. Chem. Soc. 1994, 116, 12053-4.
(23) Fraser, C.; Grubbs, R. H. Macromolecules 1995, 28, 7248-55.
(24) Nomura, K.; Schrock, R. R. Macromolecules 1996, 29, 540-5.
(25) Nomura, K.; Sakai, I.; Imanishi, Y.; Fujiki, M.; Miyamoto, Y. Macromol. Rapid Commun. 2004, 25, 571-576.
(26) Miyamoto, Y.; Fujiki, M.; Nomura, K. J. Polym. Sci., Part A: Polym. Chem. 2004, 42, 4248-4265.
(27) Manning, D. D.; Hu, X.; Beck, P.; Kiessling, L. L. J. Am. Chem. Soc. 1997, 119 3161-2.
(28) Kanai, M.; Mortell, K. H.; Kiessling, L. L. J. Am. Chem. Soc. 1997, 119, 9931-9932.
(29) Strong, L. E.; Kiessling, L. L. J. Am. Chem. Soc. 1999, 121, 6193-6196.
(30) Gordon, E. J.; Gestwicki, J. E.; Strong, L. E.; Kiessling, L. L. Chem. Biol. 2000, 7 9-16.
(31) Invest. Ophthalmol. Vis. Sci. 1998, 39, 644-648.
(32) Schnabel, W. Polymer Degradation: Principles and Practical Applications, 1993.
(33) Drouin, S. D.; Yap, G. P. A.; Fogg, D. E. Inorg. Chem. 2000, 39, 5412-5414.
(34) Drouin, S. D.; Zamanian, F.; Fogg, D. E. Organometallics 2001, 20, 5495-5497.
(35) McLain, S. J.; McCord, E. F.; Arthur, S. D.; Hauptman, E.; Feldman, J.; Nugent, W. A.; Johnson, L. K.; Mecking, S.; Brookhart, M. Polym. Mater. Sci. Eng. 1997, 76, 246-247.
(36) Bielawski, C. W.; Louie, J.; Grubbs, R. H. J. Am. Chem. Soc. 2000, 122, 12872-12873.
(37) Fogg, D. E.; dos Santos, E. N. Coord. Chem. Rev. 2004, 248, 2365-2379.
(38) Slugovc, C.; Demel, S.; Stelzer, F. Chem. Commun. 2002, 2572-2573.
(39) Love, J. A.; Sanford, M. S.; Day, M. W.; Grubbs, R. H. J. Am. Chem. Soc. 2003, 125, 10103-10109.
(40) Sanford, M. S.; Love, J. A.; Grubbs, R. H. Organometallics 2001, 20, 5314-5318.
(41) Lee, H. M.; Smith, D. C., Jr.; He, Z.; Stevens, E. D.; Yi, C. S.; Nolan, S. P. Organometallics 2001, 20, 794-797.
(42) Dharmasena, U. L.; Foucault, H. M.; dos Santos, E. N.; Fogg, D. E.; Nolan, S. P. Organometallics 2005, 24, 1056-1058.
(43) Patel, S.; Marshall, J.; Fitzke, F. W., III J. Refr. Surg. 1995, 11, 100-105.
(44) Schwab, P.; Grubbs, R. H.; Ziller, J. W. J. Am. Chem. Soc. 1996, 118, 100-10.
(45) Demel, S.; Schoefberger, W.; Slugovc, C.; Stelzer, F. J. Mol. Catal. A 2003, 200, 11-19.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A biopolymer matrix comprising a biopolymer cross-linked with a neoglycopolymer.

2. The biopolymer matrix according to claim 1, wherein said biopolymer is selected from the group consisting of: Type I collagen, Type II collagen, Type III collagen, Type IV collagen, Type V collagen, recombinant collagen, denatured collagens, gelatin, fibrin-fibrinogen, elastin, glycoprotein, alginate, chitosan, hyaluronic acid, chondroitin sulphate, glycosaminoglycan, proteoglycan, protein, or glycoprotein.

3. The biopolymer matrix according to claim 1, wherein the biopolymer is collagen.

4. The biopolymer matrix according to claim 3, wherein the matrix comprises:
(a) between about 20 and 99% by weight of water or aqueous solvent, between about 0.1 and 30% by weight of neoglycopolymer and between about 0.1 and 50% by weight of collagen;
(b) between about 60 and 99% by weight of water or aqueous solvent, between about 0.1 and 10% by weight of neoglycopolymer and between about 0.1 and 30% by weight of collagen;
(c) between about 80 and 98% by weight of water or aqueous solvent, between about 0.5 and 5% by weight of neoglycopolymer and between about 1 and 15% by weight of collagen;
(d) between about 94 and 98% by weight of water or aqueous solvent, between about 1 and 3% by weight of neoglycopolymer and between about 1 and 3% by weight of collagen; or
(e) between about 95 and 97% by weight of water or aqueous solvent, between about 1 and 2% by weight of neoglycopolymer and between about 2 and 3% by weight of collagen.

5. The biopolymer matrix according to claim 1, wherein the matrix additionally comprises a bioactive agent.

6. A method for tissue regeneration or replacement in a mammal comprising implanting or administering a biopolymer matrix according to claim 1 to said mammal.

7. The method according to claim 6, wherein said method is for treating an ophthalmic disease, disorder or injury and comprises the step of implanting or administering an ophthalmic device comprising said biopolymer matrix.

8. The method according to claim 7, wherein said ophthalmic device is an artificial cornea, a cornea replacement or corneal veneer.

9. The method according to claim 6, wherein said biopolymer matrix comprises collagen.

10. The method according to claim 7, wherein said biopolymer matrix comprises collagen.

11. The method according to claim 8, wherein said biopolymer matrix comprises collagen.

12. An ophthalmic device comprising a biopolymer matrix according to claim 1.

13. The opthalmic device according to claim 12, wherein said biopolymer matrix comprises collagen.

14. A method for producing a biopolymer matrix, comprising the step of cross-linking a biopolymer with a neoglycopolymer in an aqueous medium.

15. The method according to claim 14, wherein the cross-linking step comprises:
(a) mixing said biopolymer with said neoglycopolymer in said aqueous medium;
(b) incubating the mixture of step (a) at room temperature or physiological temperature; and
(c) removing unreacted biopolymer and neoglycopolymer from the resultant matrix.

16. The method according to claim 14, wherein said cross-linking is performed at neutral pH.

17. The method according to claim 14, wherein said biopolymer is Type I collagen, Type II collagen, Type III collagen, Type IV collagen, Type V collagen, recombinant collagen, denatured collagens, gelatin, fibrin-fibrinogen, elastin, glycoprotein, alginate, chitosan, hyaluronic acid, chondroitin sulphate, glycosaminoglycan, proteoglycan, protein, or glycoprotein.

18. The method according to claim 16, wherein said biopolymer is collagen.
